(19) United States
(12) Patent Application Publication
(10) Pub. No.: US 2025/0269185 A1
(43) Pub. Date: Aug. 28, 2025

(54) Downselector for Sensing Electrode Voltages in an Implantable Stimulator Device

(71) Applicant: Boston Scientific Neuromodulation Corporation, Valencia, CA (US)

(72) Inventor: Goran N. Marnfeldt, Valencia (CA)

(21) Appl. No.: 19/055,361

(22) Filed: Feb. 17, 2025

Related U.S. Application Data
(60) Provisional application No. 63/557,931, filed on Feb. 26, 2024.

(51) Int. Cl. A61N 1/36; A61N 1/20

(52) U.S. Cl. CPC A61N 1/36139; A61N 1/20; A61N 1/36125

(57) ABSTRACT

Sensing circuitry for an implantable stimulator device is disclosed. The sensing circuitry includes a downselector with switching networks used to couple selected electrode nodes and/or DC voltages to one or more sense amps. The switching networks comprise high-voltage devices that are controlled in accordance with a high-voltage power supply voltage VH. VH may be generated by a boost circuit, and may be set to the higher of a boosted voltage and a compliance voltage that powers the stimulation circuitry. The switching networks include three switches, some of which may comprise transfer gates. The downselector can be controlled to couple one or more electrodes nodes to a particular sense amp during sensing durations. The downselector can also be controlled during stimulation durations to couple one or more electrodes nodes together, thus supplementing operation of the stimulation circuitry.

### CROSS REFERENCE TO RELATED APPLICATIONS

This is a non-provisional application of U.S. Provisional Patent Application Ser. No. 63/557,931, filed Feb. 26, 2024, which is incorporated herein by reference, and to which priority is claimed.

### FIELD OF THE INVENTION

This application relates to Implantable Medical Devices (IMDs), and more specifically to circuitry to assist with sensing neural responses to stimulation in an implantable stimulator device.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) or Deep Brain Stimulation (DBS) system. However, the present invention may find applicability with any stimulator device system.

A stimulator system typically includes an Implantable Pulse Generator (IPG) 10 like that shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12, or some conductive portion of the case, can also comprise an electrode (Ec). In an SCS application, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. In a DBS application, the electrode leads are implanted in the brain through holes in the skull, and lead extension are used to connect the leads to the IPG which is typically implanted under the clavicle (collarbone). In other IPG examples designed for implantation directly at a site requiring stimulation, the IPG can be lead-less, having electrodes 16 instead appearing on the body of the IPG 10 for contacting the patient's tissue. The IPG lead(s) can be integrated with and permanently connected to the IPG 10 in other solutions. SCS therapy can relieve symptoms such as chronic back pain, while DBS therapy can alleviate Parkinsonian symptoms such as tremor and rigidity. IPG 10 as described should be understood as including External Trial Stimulators (ETSs), which mimic operation of the IPG 10 during trials periods when leads have been implanted in the patient but the IPG 10 has not. Sec, e.g., U.S. Pat. No. 9,259,574 (disclosing an ETS).

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30x), as shown in the example of FIG. 2A. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as an anode (during its first phase 30a), and thus provides pulses which source a positive current of amplitude +I to the tissue. Electrode E2 has been selected as a cathode (again during first phase 30a), and thus provides pulses which sink a corresponding negative current of amplitude −I from the tissue. This is an example of bipolar stimulation, in which the electrode array 17 includes one anode pole and one cathode pole. Note that more than one electrode on the lead may be selected to act as an anode electrode to form an anode pole at a given time, and more than one electrode may be selected to act as a cathode to form a cathode pole at a given time, as explained further in U.S. Pat. No. 10,881,859. Stimulation provided by the IPG 10 can also be monopolar. In monopolar stimulation, the electrode array is programmed with a single pole of a given polarity (e.g., a cathode pole), with the conductive case electrode Ec acting as a return (e.g., an anode pole). Again, more than one electrode on the lead may be active to form the pole during monopolar stimulation.

IPG 10 as mentioned includes stimulation circuitry 28 to form prescribed stimulation at a patient's tissue. FIG. 3 shows an example of stimulation circuitry 28, which includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs in the stimulation circuitry 28 allows any of the electrodes 16 to act as anodes or cathodes to create a current through a patient's tissue, R, hopefully with good therapeutic effect. Consistent with the example provided in FIG. 2A, FIG. 3 shows operation during the first phase 30a in which electrode E1 has been selected as an anode electrode to source current I to the tissue R and E2 has been selected as a cathode electrode to sink current from the tissue. Thus PDAC1 and NDAC2 are digitally programmed (via busses <Ip1>, <In2>) to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16. Other stimulation circuitries 28 can also be used in the IPG 10, including ones that includes switching matrices between the electrode nodes ci 39 and the N/PDACs. Sec, e.g., U.S. Pat. Nos. 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as IPG master control circuitry 102 (see FIG. 5), telemetry circuitry (for interfacing off chip with telemetry antennas 27a and/or 27b), circuitry for generating the compliance voltage Vcomp (as explained next), various measurement circuits, etc.

Power for the stimulation circuitry 28 is provided by a compliance voltage Vcomp, as described in further detail in U.S. Pat. No. 11,040,202 and U.S. Patent Application Publications 2013/0289665, 2018/0071520, and 2021/0046120. The compliance voltage Vcomp may be coupled to the source circuitry (e.g., the PDAC(s)), while ground may be coupled to the sink circuitry (e.g., the NDAC(s)), such that the stimulation circuitry 28 is powered by Vcomp and ground. Other power supply voltages may be used with the PDACs and NDACs, but these aren't shown in FIG. 3 for simplicity. Preferably, the compliance voltage Vcomp can be produced by a Vcomp regulator 49. Vcomp regulator 49 receives the voltage of the battery 14 (Vbat) and boosts this voltage to a higher value required for the compliance voltage Vcomp. Vcomp regulator 49 can comprise an inductor-based boost converter or a capacitor-based charge pump for example. The regulator 49 can vary the value of Vcomp based on measurements taken from the stimulation circuitry 28, such as the voltage drops across the active DACs in the stimulation circuitry 28. Using such measurements allows Vcomp to be established at an energy-efficient level: high enough to form the prescribed current without loading (i.e., without producing less current that prescribed), yet low enough to not needlessly waste power in the stimulation circuitry 28 when forming the prescribed current. In this respect, Vcomp can be variable, and typically ranges from about 5 to 15 Volts.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. While useful, DC-blocking capacitors 38 are not strictly required in all IPG designs and applications.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will (primarily) build up across the DC-blockings capacitors C1 and C2 associated with the electrodes E1 and E2 used to produce the current, giving rise to voltages Vc1 and Vc2 (I=C*dV/dt). During the second pulse phase 30b, when the polarity of the current I is reversed at the selected electrodes E1 and E2, the stored charge on capacitors C1 and C2 is recovered, and thus voltages Vc1 and Vc2 hopefully return to 0V at the end the second pulse phase 30b.

Charge recovery using phases 30a and 30b is said to be “active” because the P/NDACs in stimulation circuitry 28 actively drive a current, in particular during the last phase 30b to recover charge stored after the first phase 30a. However, such active charge recovery may not be perfect, and some residual charge may be present in capacitive structures even after phase 30b is completed. Accordingly, the stimulation circuitry 28 can also provide for passive charge recovery. Passive charge recovery is implemented using passive charge recovery switches PRi 41 as shown in FIG. 3. These switches 41 when selected via assertion of control signals <Xi> couple each electrode node ei to a passive recovery voltage Vpr established on bus 43. As explained in U.S. Pat. Nos. 10,716,937 and 10,792,491, this allows any stored charge to be recovered through the patient's tissue, R. Control signals <Xi> are usually asserted to cause passive charge recovery after each pulse (e.g., after each last phase 30b) during periods 30c shown in FIG. 2A. Because passive charge recovery involves capacitive discharge through the resistance R of the patient's tissue, such discharge manifests as an exponential decay in current, as shown in FIG. 2A. As also discussed in the '937 patent, each of the passive charge recovery switches 41 can be associated with a variable resistance, and as such each switch 41 can be controlled by a bus of signals <Xi> to control the resistance at which passive charge recovery occurs—i.e., the on resistance of the switches 41 when they are closed. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, various sensed responses as discussed further below, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 27a in the IPG 10. The external controller 60 can also have a far-field RF antenna 64b capable of wirelessly communicating with the RF antenna 27b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 27a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 27b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

A stimulator device is disclosed, which may comprise: a plurality of electrode nodes, wherein each of the electrode nodes is associated with a different electrode configured to contact a patient's tissue; at least one sense amplifier comprising a first input and configured to sense a signal from the patient's tissue; and a plurality of first switching networks each comprising a first switch and a second switch connected in series between one of electrode nodes and the first input, and a third switch connected between a reference voltage and a node between the first and second switches, wherein each of the first switching networks is controllable by at least one first control signal.

In one example, each at least one first control signal comprises a first signal and a second signal, wherein the second signal is a logical complement of the first signal. In one example, the first signal controls the first and second switches, and wherein the second signal controls the third switch. In one example, the first switch comprises a first transfer gate, and wherein the second switch comprises a second transfer gate. In one example, the first and second transfers gates each comprise a N-channel and P-channel transistor connected in parallel. In one example, the first signal controls the N-channel transistors in the first and second transfer gates, and wherein the second signal controls the P-channel transistors in the first and second transfer gates. In one example, the second signal also controls and the third switch. In one example, the reference voltage comprises ground. In one example, each first switching network is configured so that the first and second switches are closed at the same time that the third switch is opened, and the first and second switches are opened at the same time that the third switch is closed. In one example, the at least one first control signals are issued by control circuitry. In one example, the control circuitry is powered by a first power supply voltage. In one example, the at least one first control signals are established in a second domain comprising a second power supply voltage higher than the first power supply voltage. In one example, the stimulator device further comprises level translator circuitry to produce the at least one first control signals from control signals established in a first domain comprising the first power supply voltage. In one example, the stimulator device further comprises a boost circuit configured to generate a boosted power supply voltage higher than the first power supply voltage. In one example, the second power supply voltage comprises the boosted power supply voltage. In one example, the stimulator device further comprises stimulation circuitry configured to issue stimulation to the plurality of electrode nodes, wherein the stimulation circuitry is powered by a compliance power supply voltage. In one example, the stimulator device further comprises a selector circuit configured to establish the second power supply voltage at a higher of the boosted and compliance power supply voltages. In one example, the at least one sense amplifier comprises a second input, and further comprising a plurality of second switching networks each comprising a first switch and a second switch connected in series between the second input and either (i) one of the electrode nodes, or (ii) a DC voltage, and a third switch connected between a reference voltage and a node between the first and second switches, wherein each of the second switching networks is controllable by at least one second control signal. In one example, the stimulator device further comprises control circuitry configured to issue the at least one first control signals and the at least one second control signals. In one example, the control circuitry is configured to issue the at least one first control signals and the at least one second control signals to couple different of the electrode nodes to the first and second inputs. In one example, the control circuitry is configured to issue the at least one first control signals and the at least one second control signals to couple one of the electrode nodes to the first input and the DC voltage to the second input.

A stimulator device is disclosed, which may comprise: a plurality of electrode nodes, wherein each of the electrode nodes is associated with a different electrode configured to contact a patient's tissue; at least one sense amplifier comprising a first input and configured to sense a signal from the patient's tissue during sensing durations; stimulation circuitry configured to issue stimulation to selected electrode nodes during stimulation durations; and a plurality of first switching networks controllable by first control signals to: connect two or more of the electrode nodes together and to the first input during the sensing durations, and connect two or more of the selected electrode nodes together during the stimulation durations.

In one example, one of the first switching networks is connected between each of electrode nodes and the first input. In one example, each of the plurality of first switching networks comprises a first switch and a second switch connected in series between one of electrode nodes and the first input, and a third switch connected between a reference voltage and a node between the first and second switches. In one example, each of the first switching networks is controllable by a first and second of the first control signals. In one example, the second signal is a logical complement of the first signal. In one example, each first switching network is configured so that the first and second switches are closed at the same time that the third switch is opened, and the first and second switches are opened at the same time that the third switch is closed. In one example, the first switching networks at the two or more electrodes are closed during the stimulation durations to connect the two or more electrode nodes together. In one example, the first switching networks at the two or more electrodes are closed to connect the two or more electrode nodes together and to the first input during the sensing durations. In one example, the at least one sense amplifier is enabled during the sensing durations, and is disabled during the stimulation durations. In one example, the stimulator circuitry comprises a programmable source circuit and a sink circuit dedicated and connected to each electrode node. In one example, at least one of the source circuits and at least one of the sink circuits is programmed to provide the stimulation to the selected electrode nodes. during the stimulation durations. In one example, the at least one first control signals are issued by control circuitry. In one example, the control circuitry is powered by a first power supply voltage. In one example, the at least one first control signals are established in a second domain comprising a second power supply voltage higher than the first power supply voltage. In one example, the stimulator device further comprises a boost circuit to generate the second power supply voltage. In one example, the stimulator device further comprises a lead, wherein at least some of the electrodes comprise split-ring electrodes on the lead. In one example, the selected electrode nodes are associated with the split-ring electrodes at a common longitudinal position on the lead. In one example, the at least one sense amplifier comprises a second input, and further comprising a plurality of second switching networks controllable by second control signals to: connect two or more different of the electrode nodes together and to the second input during the sensing durations, and connect two or more different of the selected electrode nodes together during the stimulation durations.

### BRIEF DESCRIPTION OF THE DRAWINGS

FIG. 1 shows an Implantable Pulse Generator (IPG), in accordance with the prior art.

FIGS. 2A and 2B show an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5 shows an IPG having sensing circuitry.

FIG. 6 shows details of the downselector in the sensing circuitry which is used (inter alia) to connect various electrode nodes and DC voltages to the inputs of the sense amps.

FIGS. 7A and 7B shows switching networks used in the downselector.

FIGS. 8 and 9 show circuitry for generating a high-voltage power supply for use in controlling the switching networks.

FIG. 10 shows generation of control signals for the switching networks using the high-voltage power supply.

FIG. 11 shows control of the downselector during sensing to combine sensing electrode nodes together.

FIGS. 12A and 12B show control of the downselector during stimulation to combine stimulation electrode nodes together.

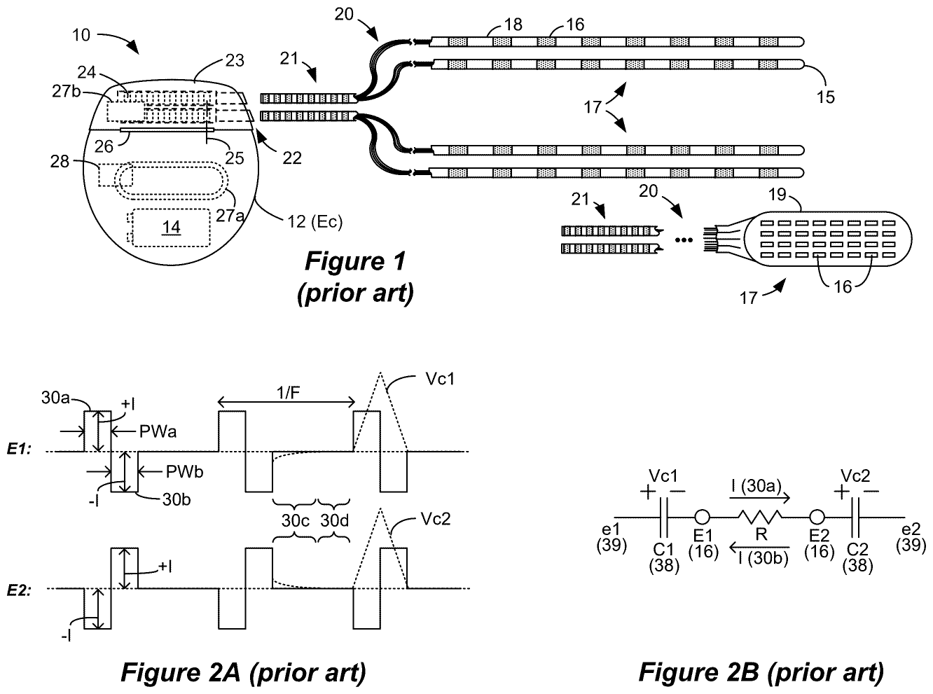

### DETAILED DESCRIPTION

An increasingly interesting development in pulse generator systems is the addition of sensing capability to complement the stimulation that such systems provide. For example, and as explained in U.S. Patent Application Publication 2017/0296823, it can be beneficial to sense a neural response produced by neural tissue that has received stimulation from an IPG. U.S. Patent Application Publication 2017/0296823 shows an example where sensing of neural responses is useful in an SCS context, and in particular discusses the sensing of Evoked Compound Action Potentials, or “ECAPs.” U.S. Patent Application Publication 2022/0040486 shows an example where sensing of neural responses is useful in a DBS context, and in particular discusses the sensing of Evoked Resonant Neural Activity, or “ERNA.” Sensing can also be used to sense local field potentials (LFPs) and other voltages in the tissue, such as stimulation artifacts. Sec, e.g., U.S. Patent Application Publication 2022/0323764.

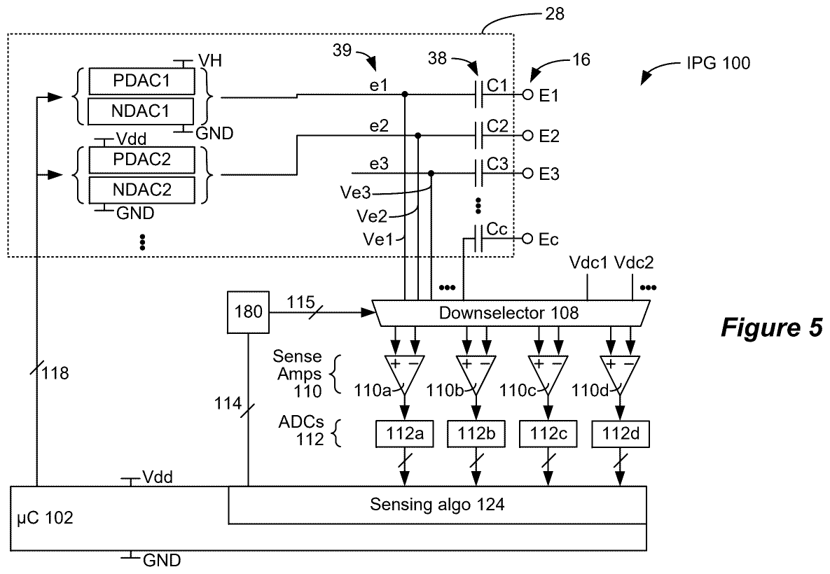

FIG. 5 shows basic circuitry for an IPG 100 having sensing capability. The IPG 100 includes control circuitry 102, which may comprise a microcontroller for example, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs) in the IPG 10 as described earlier, which ASIC(s) may additionally include the other circuitry shown in FIG. 5. The control circuitry may be powered by a low-voltage digital power supply Vdd, which may generally comprise 1.8 Volts in one example.

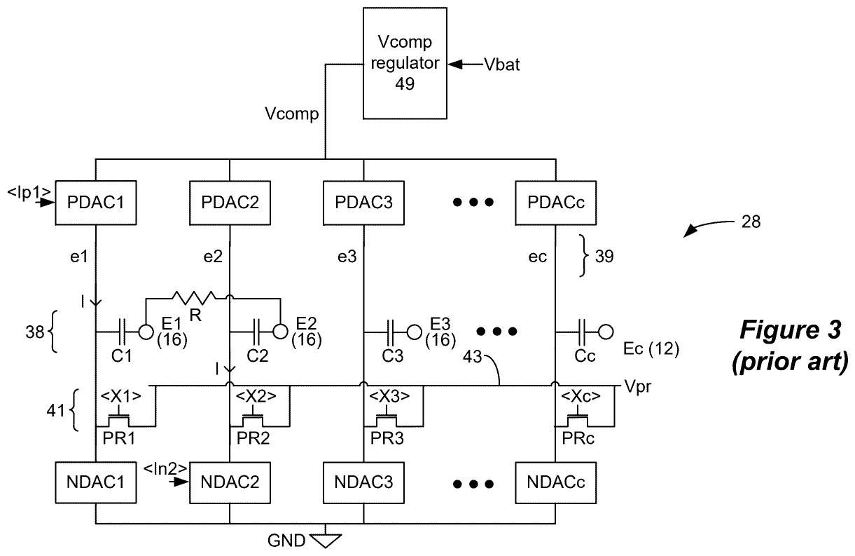

FIG. 5 includes the stimulation circuitry 28 described earlier (FIG. 3), including one or more DACs (PDACs and NDACs). A bus 118 provides digital control signals to the DACs to produce currents or voltages of prescribed amplitudes and with the correct timing at the electrodes selected for stimulation. The current paths to the electrodes include the DC-blocking capacitors 38 described earlier, which separate the electrode nodes 39 from the electrodes 16.

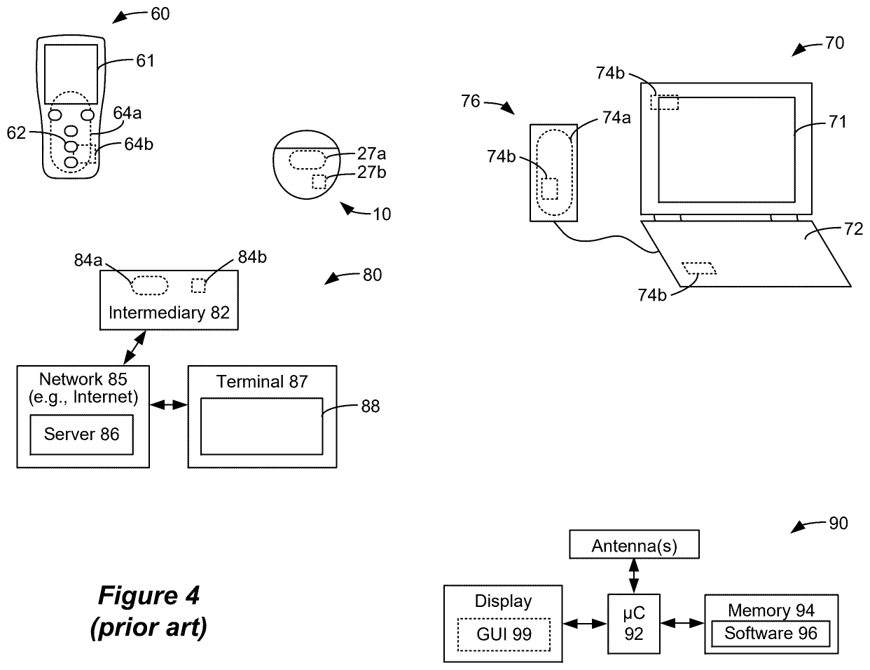

FIG. 5 also shows circuitry used for sensing. As shown, the electrode nodes 39 are input to a downselector (MUX) 108. The downselector 108 comprises a multiplexer controlled by a bus 115. The signals on bus 115 may comprise level shifted (180) versions of signals issued via a bus 114 from the control circuitry 102, as discussed further below with reference to FIG. 10. As explained further below, the downselector 108 can be controlled in different manners and to different useful ends, but in FIG. 5 it is assumed that the downselector 108 is controlled by bus 115 to select one or more sensing electrodes. The downselector 108 can also operate to select one or more DC voltages (Vdc1, Vdc2, etc.), as is useful in single-ended sensing as discussed below. The DC voltages can comprise any DC voltage produced within the IPG, such as ground, the voltage of the battery (Vbat), the compliance voltage Vcomp, a power supply voltage (Vdd), or some fraction of these (such as Vcomp/2 or Vdd/2). The sensing electrode(s) and/or DC voltages selected via bus 115 can be determined automatically by control circuitry 102 and/or a sensing algorithm 124, as described further below. However, the sensing electrode(s) may also be selected by the user (e.g., a clinician) via an external system 60, 70 or 80 (FIG. 4).

Electrodes nodes 39 and/or DC voltages selected are provided by the downselector 108 to sense amplifier circuitry 110 comprising one or more sense amps 110x, each having a positive (+) and negative (−) input. In the example shown, there are four sense amps 110a-110d, which allows sensing to occur on four sensing channels simultaneously. Other implementations may use only a single sense amp. Sensing can occur differentially using two sensing electrodes (two selected electrode nodes 39) that are provided to the positive and negative inputs of a sense amp, or in a single-ended fashion using a single sensing electrode (positive input) and one of the DC voltages (negative input). Differential sensing can be useful to cancel any common mode voltages present in the tissue and reflected at the electrodes, such as voltages created by the stimulation itself. Sec, e.g., U.S. Patent Application Publication 2021/0236829.

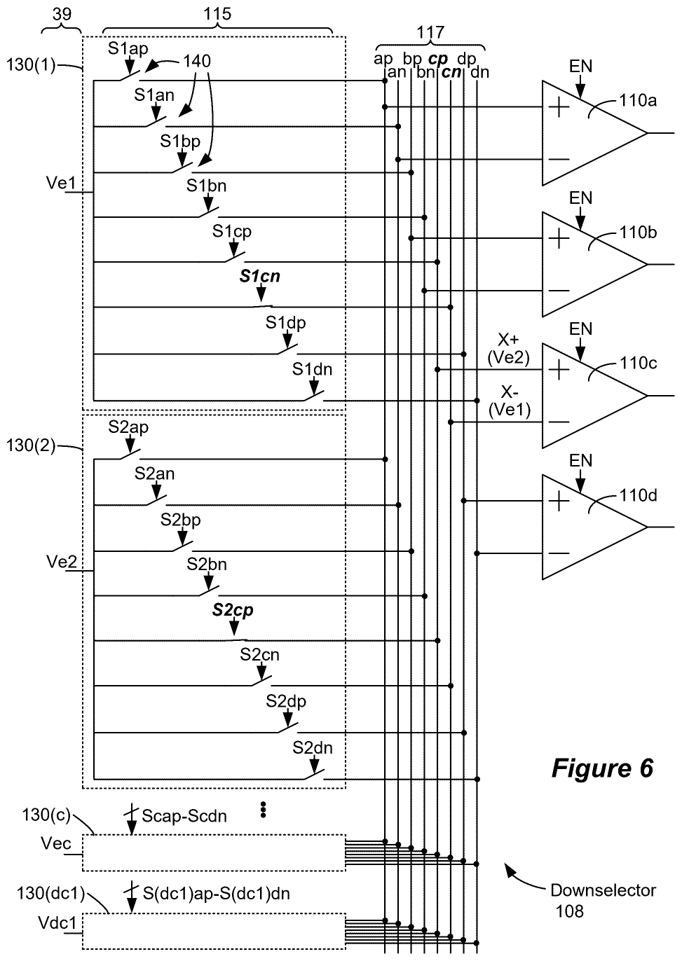

Further details of circuitry useable for the sense amps is disclosed in U.S. Pat. No. 11,633,138 and U.S. Patent Application Publication 2023/0173273, which are incorporated herein by reference in their entireties. Although not shown, but as described in these references, the sense amps 110a-d may include one or more differential amplifiers whose gains are fixed or programmable, and may also include front-end circuitry at its inputs and/or back-end circuitry at its outputs. Such front- or back-end circuitry of the sense amps 110a-d may include filters, track-and-hold circuitry, attenuators, input and output voltage protection, voltage assessment logic, and the like. The timing at which sensing occurs can be affected by a sensing enable signal (EN, FIG. 6) provided to the sense amps 110x.

The analog waveform(s) output by the sense amps 110a-110d are preferably converted to digital signals by one or more Analog-to-Digital converters (ADC) 112a-d, and input to the IPG's control circuitry 102. The ADCs 112 can be included within the control circuitry 102's input stage as well. The control circuitry 102 can be programmed with a sensing algorithm 124 to evaluate the sensed signals, and to take appropriate actions as a result. For example, the sensing algorithm 124 may change the stimulation in accordance with a sensed neural response, such as an ECAP or ERNA signal, and can issue new control signals via bus 118 to change operation of the stimulation circuitry 28 to affect better treatment for the patient. The sensing algorithm 124 may also cause the selection of new sensing electrode(s), which can be affected by issuing new control signals on busses 114 and 115. Selecting optimal sensing electrode(s) can be important, and may be determined in light of stimulation that is being provided. In this regard, sensing electrodes may be selected near enough to the electrodes providing stimulation (e.g., E1 and E2) to allow for proper sensing, but far enough from the stimulation that the stimulation doesn't substantially interfere with sensing. Sec, e.g., U.S. Patent Application Publication 2020/0155019.

FIG. 6 shows further details of the downselector 108. The downselector 108 includes a number of different switch matrices 130 for each electrode node 39 (e.g., 130(1) for Vel, 130(2) for Ve2, . . . , 130(c) for case electrode Vec) as well as a switch matrix for each DC voltage (e.g., 130(dc1) for Vdc1). Each switch matrix 130 has a number of switches 140 which may be configured as switch networks as described shortly. Each switch matrix 130 preferably includes as many switch networks 140 as there are sense amplifier inputs, for example, eight, because each sense amp 110a-110d has two inputs (+ and −). Each switch network 140 is controlled by one or more control signals on bus 115 as discussed further below.

Through this arrangement, a given electrode node (or DC voltage) can be connected to any input on any of the sense amps 110x. For example, signal S1ap connects Vel (1) to sense amp 110a's (a) positive input (p); signal S1an connects Vel (1) to sense amp 110a's (a) negative input (n); signal S1bp connects Vel (1) to sense amp 110b's (b) positive input (p); and so on. The connection of these voltages to the sense amps 110x can occur using a number of crossbars 117. For example, crossbar ‘ap’ connects the ‘ap’ switches (S1ap, S2ap, etc.) to the positive input of sense amp 110a. FIG. 6 shows an example in which control signals S1en and S2cp are asserted, which connects the voltage at electrode node e2 (Ve2) to the positive input (X+) of sense amp 110c via crossbar cp, and the voltage at electrode node e1 (Vel) to the negative input (X−) of sense amp 110c via crossbar cn. This allows sense amp 110c to sense tissue signals at electrode E2 using electrode E1 as a reference. The downselector 108 as designed and controlled provides great flexibility, because each sense amp 110x can be controlled (in different sensing channels) to sense between a wide combination of potentially relevant voltages.

The voltages being input to the sense amps 110a-d may relatively large even if signals of interest are quite small. For example, an ECAP that is desirable to sense may comprise a small-amplitude AC signals on the order of micro Volts or milliVolts. However, the stimulation itself may provide voltages to the tissue (e.g., stimulation artifacts) that are much larger, on the order of several of Volts. The ECAP of interest may coincide in time with these larger tissue signals, and thus sensing the ECAP may require providing large voltages to the sense amps 110x. More particularly, because the stimulation circuitry 28 used to provide the stimulation is powered using supply voltages Vcomp (which may be large) and ground (see FIG. 3), voltages in the tissue may vary between these voltages.

Because the tissue voltages being passed by the switching networks 140 in the downselector 108 may be large, it is advisable to construct the switching networks 140 from high voltage devices. Therefore, transistors in the switching networks 140 may comprise high voltage transistors capable of handling higher voltages. (For example, they may have thicker gate oxides than do normal logic transistors operable at Vdd and used to form digital logic circuits). In one example, and because the switching networks 140 may pass voltages referenced to the compliance voltage Vcomp, the devices in the switching networks 140 can comprise devices capable of operating at Vcomp.

However, simply operating the switching networks 140 in the Vcomp power domain can have drawbacks. First, Vcomp as discussed above can vary during operation of the IPG 100, being higher at certain points (e.g., when higher amplitude pulses are being provided) and lower at other points (e.g., lower amplitude pulses). As such, it may not be advisable to control the switching networks 140 using Vcomp. For example, if Vcomp is provided to gates of transistors in the switching network 140, the on resistances of these transistors can vary, meaning that the tissues signals will be passed to the sense amps 110a-110d through a variable resistance. This can affect the reliability at which signals are sensed.

Further, simply providing a single switch (a single transistor) in each switching network 140 has drawbacks as well. Transistors are non-ideal, and in their off state can have significant resistances and capacitances. Thus, even when the switching networks 140 are off, the tissue signals could leak through the transistor to some degree and be presented to the inputs of the sense amps 110x. This could affect (e.g., unbalance) the DC level of the voltages at the sense amps, and their subsequent ability to sense signals of interest.

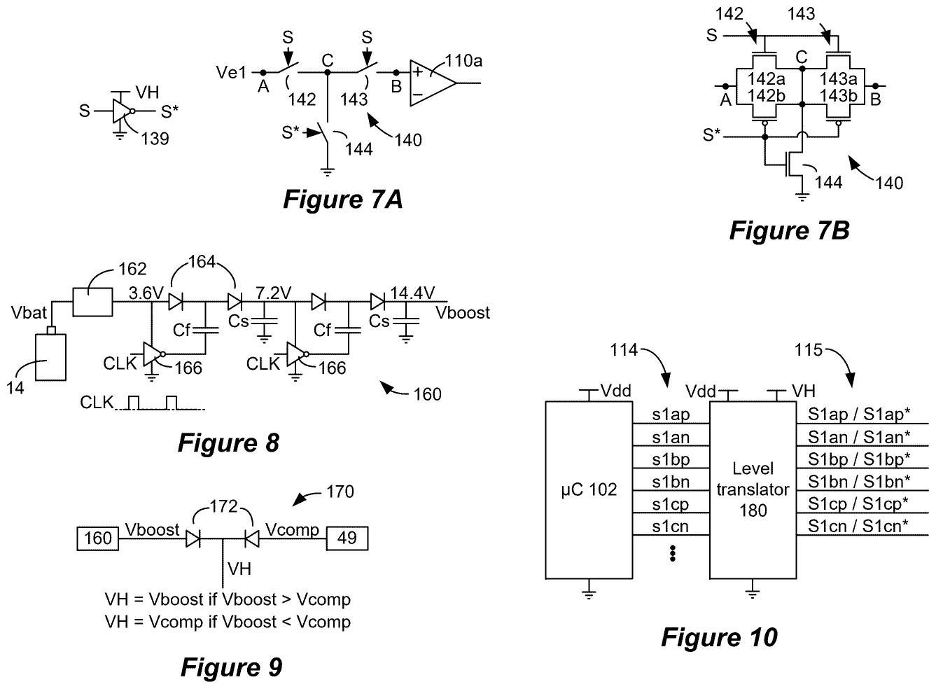

To address these concerns, the inventors have devised improved switching networks 140 for use in the downselector 108 of an IPG 100, as well as non-conventional use of the downselector 197 during sensing and the provisional of stimulation. FIG. 7A shows the switching network 140 in a functional manner as comprising three switches 142, 143, and 144. Switch 142 is coupled between the electrode node 39 (or DC voltage) (node A) and a node C; switch 143 is coupled between the relevant crossbar 117 to the sense amps 110x (node B) and node C. Node C between the two switches 142 and 143 is couplable to ground through switch 144. The switches 142-144 are controlled by a control signal and its logical complement. In FIG. 7A, these control signals are denoted generically as S and S*, but there would be unique control signals provided for each switching network 140 (e.g., S1ap and S1ap*, S1an and S1an*, etc.) as shown in FIG. 10. Control signals S* can be formed by high-voltage inverters 139 powered by a high-voltage power supply VH explained further below.

Switches 142 and 143 are controlled in unison to be closed (on) when S is high (‘1’), and to be opened (off) when S is low (‘0’). When S is high, S* is low and switch 144 is opened (off) thus isolating node C from ground. This connects nodes A and B, and thus connects the voltage to be sensed to the relevant crossbar 117/sense amp 110x. When S is low, switches 142 and 143 are opened (off) which decouples node A from node B. S* is high, which closes switch 144 (on) and grounds node C. While not strictly necessary, use of switch 144 is preferred to prevent any signal coupling between nodes A and B when switches 142 and 143 are closed. This is beneficial because as noted above switches 142 and 143 may have residual resistances and capacitances that could promote signal leakage between nodes A and B even when these switches are closed. Although switch 144 is depicted as coupling to node C to ground in FIG. 7A, node C could also be coupled to any other reference voltage which may be different from ground.

FIG. 7B shows a preferred implementation of the switching network 140 at a transistor level. Here, switches 142 and 143 are configured as transfer gates, with switch 142 comprising transistors 142a (N-channel) and 142b (P-channel) connected in parallel between nodes A and C, and with switch 143 comprising transistors 143a (N-channel) and 143b (P-channel) connected in parallel between nodes B and C. The N-channel transistors 142a and 143a are controlled by S, while P-channel transistors 142b and 143b and N-channel transistor 144 are controlled by S*. Thus, when S is high, transistors 142a and 143a are closed, and S* being low will likewise close transistors 142b and 143b to couple nodes A and B. Use of transfer gates 142a/142b and 143a/143b are preferred to more accurately pass the voltage from node A to B regardless of its magnitude. S* being low will open N-channel transistor 144 to isolate node C from ground. By contrast, when S is low and S* is high, transistors 142a, 143a, 142b, and 143b are opened to isolate nodes A and B. S* being high will close transistor 144 to ground node C, which as discussed above prevents signal leakage between A and B.

The switching networks 140 are preferably formed from high-voltage components, and are likewise operated at high voltages as necessary for passing the potentially high voltage tissue signals. To do so, the control signals are established in a high-voltage power supply domain in accordance with a high-voltage power supply VH, the generation of which is described in FIGS. 8 and 9. VH is preferably set to at least equal to Vboost, which is generated using boost circuit 160 of FIG. 8. Alternatively, if the selector circuit 170 of FIG. 9 is used, VH can be set to Vcomp—the power supply voltage of the stimulation circuitry 28 (see FIG. 3)—if Vcomp is larger than Vboost.

The boost circuit 160 generates Vboost from the battery voltage Vbat, which may be regulated via a regulator 162. In the example shown, the regulated battery voltage is assumed to be 3.6V, which is doubled twice in two stages to form Vboost=14.4V. Boosting is accomplished in this example using a two-stage Dickson charge pump comprising a series of diodes 164, fly and storage capacitors Cf and Cs, and a clock CLK. The design of this charge pump is only briefly explained. 3.6V passes through a first diode 164, which is boosted by the first fly capacitor Cf to 7.2 in a first stage when the clock is low. This boosted voltage passes through a second diode and is stored on a first storage capacitor Cs, which cannot pass backwards through the second diode even after the clock returns high. This process and circuitry is essentially repeated in a second stage to boost 7.2 to 14.4V as desired. Boosting circuit 160 is just one example, and other forms of voltage boosting circuitry could be used, including those that are inductor based. Vboost may also be set to different voltage values.

Vboost is preferably set to be close to potential magnitudes of tissue voltages (e.g., on the order of Vcomp), which as noted earlier are referenced to Vcomp and can be as high as 15V. Setting Vboost high ensures that the control signals for the switching networks 140 will be high enough to properly and accurately pass tissue signals through the switches 142 and 143. However, because Vcomp (which is variable) may be higher than Vboost from time to time, an optional selector circuit 170 can be used to set power supply VH to the higher of the two, as shown in FIG. 9. Selector circuit 170 passes Vboost as generated by boost circuit 160 and the compliance voltage Vcomp as generated by the compliance voltage generator 49 (FIG. 3) through forward-biased Schottky diodes 172 to node VH. This sets VH to the higher of the two voltages. In other words, VH is set to a minimum value of Vboost (if Vboost>Vcomp), but VH will also equal Vcomp if Vcomp>Vboost. As noted, selector circuit 170 is optional, and other examples VH can simply be set to Vboost.

Once VH has been generated, it can be used to set the voltages of the control signals accordingly, and this can occur using level translator circuitry 180 as shown in FIG. 10. FIG. 10 shows the issuance of downselector control signals from the control circuitry 102, which are issued in a low-voltage (Vdd) digital domain (e.g., ‘0’=0V, ‘1’=Vdd) on bus 114. These low-voltage control signals are denoted with a lower-case “s” to differentiate them from high-voltage domain versions of these signals denoted with an upper-case “S.” These low-voltage control signals are provided to level translator circuitry 180 to convert them from a low-voltage (Vdd) to a high-voltage (VH) digital domain (e.g., ‘0’=0V, ‘1’=VH) on bus 115. Such level translator circuitry 108 is well known, and may include high-voltage inverters 139 (FIG. 7A) to produce both high-voltage control signals (e.g., S1ap) and their complements (e.g., S1ap*) referenced to VH. The level translator circuitry 180 can be considered part of the control circuitry.

The downselector 108 provides flexibility in the selection of which electrodes will operate as sensing electrodes to sense tissue signals. Conventionally, the downselector 108 would be controlled via control signals S/S* to allowing sensing of tissue signals at one electrode relative to another electrode (or relative to a DC voltage). FIG. 6 illustrates such a conventional use, in which tissue signals at electrode E2 are sensed relative to electrode E1.

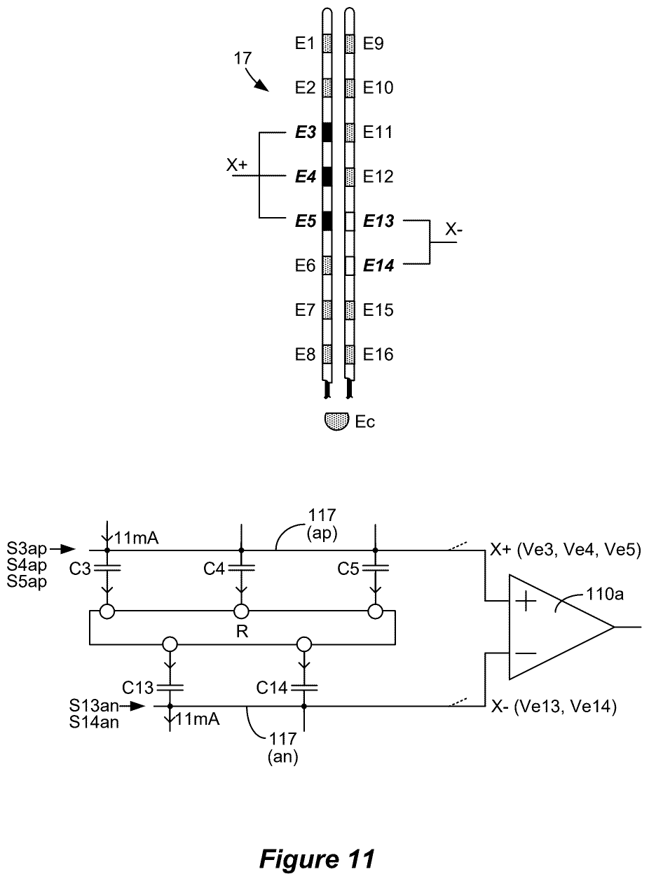

The downselector 108 may also be controlled to allow a plurality of electrodes to be connected together to effectively operate as a single sensing electrode at a given time, and FIG. 11 shows an example. In this example, sensing occurs at electrodes E3, E4, and E5, with the electrode nodes 39 at these electrodes being connected together within the downselector 108. The combined sensed tissue signals at these electrodes (Ve3, Ve4, Ve5) are provided to the positive input of sense amp 110a (X+). The electrode nodes for electrodes E13 and E14 are similarly connected to allow these electrodes to act as a sensing reference, with their combined signal (Ve13 and Ve14) being provided to the negative input of sense amp 110a (X−). In effect, electrodes E3-E5 act as a single sensing electrode, and electrodes E13 and E14 act as a single sensing reference.

How the downselector 108 is controlled to achieve this result is shown in FIG. 11. To connect the electrode nodes at electrodes E3-E5, control signals S3ap, S4ap, and S5ap are asserted. This closes the switching networks 140 at the corresponding electrode nodes (Ve3, Ve4, Ve5) to present this combined signal to crossbar ap, which in turn connects this combined sensed signal to the positive input of sense amp 110a. (The switching networks 140 are not shown in FIG. 11 for simplicity). Of course, these combined electrode nodes signals could be connected to any available crossbar 117 and sense amp 110x (such as 110b, meaning S3bp, S4bp, and S5bp would be asserted). Likewise, control signals S13an and S14an are asserted to close the switching networks 140 at the corresponding electrode nodes (Ve13 and Ve14) to present this combined signal to crossbar an, which in turn connects this combined sensed signal to the negative input (n) of sense amp 110a. In short, the downselector 108 may be controlled such that any number of electrodes (including the case electrode) can act in unison as sensing electrodes.

Typically, the control circuitry 102 will assert the downselector control signals S during durations when a signal is to be sensed at the sense amps 110x, at which time the sense amps 110x may also be enabled (EN=‘1’; FIG. 6). During all other periods—such as during periods when stimulation pulses are being delivered—the control circuit 102 may deassert all control signals (S =‘0’) to open all of the switching networks 140 to decouple the electrode nodes (and DC voltages) (A) from the sense amps (B). The sense amps 110x may also be disabled at these times.

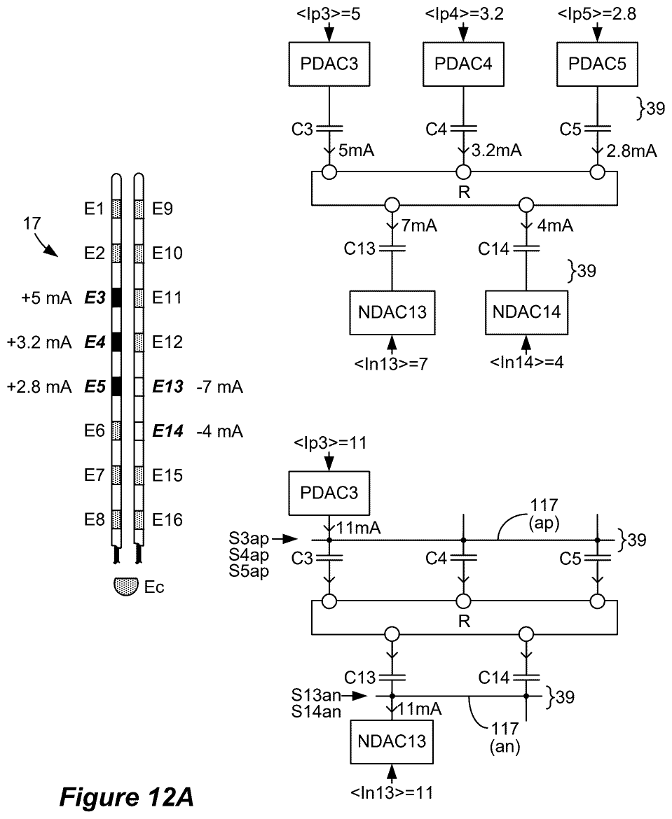

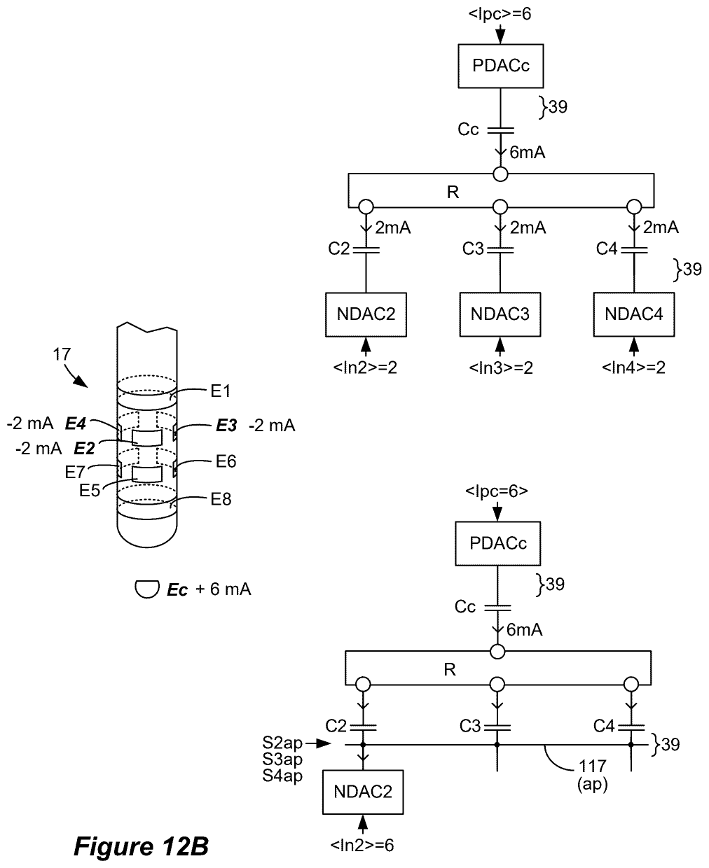

That being said, switching networks 140 in the downselector 108 can also be closed during stimulation to useful effect and to provide stimulation to the tissue in manners different than what the stimulation circuitry 28 could provide alone (FIG. 3). In this regard, the downselector 108 may be controlled to connect one or more of the electrodes together during stimulation, and this is shown in a first example in FIG. 12A. In FIG. 12A, it is assumed that more than two electrodes will provide stimulation to the tissue R at a given time. Ideally, it is assumed that anodic current will be sourced to the tissue from anodic electrodes E3 (+5 mA), E4 (+3.2 mA), and E5 (+2.8 mA), and that cathodic current will be sunk from the tissue at cathodic electrodes E13 (−7 mA) and E14 (−4 mA). In sum, electrodes E3-E5 thus ideally provide an anodic current of +11 mA, while electrodes E13 and E14 ideally provide a corresponding cathodic current of −11 mA.

The top of FIG. 12A shows the traditional manner in which stimulation circuitry 28 would provide these currents to the tissue R without use of the downselector 108. The relevant PDACs or NDACs at the selected electrodes would be digitally programmed to produce the ideal amount of current (e.g., <Ip3> provides +5 mA at electrode E3, <In13> provides −7 mA of current at electrode E13, etc.). Because the downselector 108 is not active, all control signals to the various switching networks 140 are deasserted (S=‘0’) and all switching networks 140 are open.

The bottom of FIG. 12A shows an alterative method for approximately delivering this stimulation in a manner that involves control of the downselector 108. Here, a single PDAC at one of the anodic electrodes is programmed to provide the desired summed anodic current of +11 mA, and a single NDAC at one of the cathodic electrodes is programmed to provide the desired summed cathodic current of −11 mA. As depicted, PDAC3 provides the summed anodic current, although one or more of PDAC4 and PDAC5 could also provide part or all of this current. Likewise, NDAC13 provides the summed cathodic current but NDAC 14 could also provide part of all of this current.

The downselector 108 is then controlled to connect the anodic electrodes nodes together (E3-E5) so that they in sum issue the summed anodic current to the tissue, and to connect the cathodic electrodes nodes together (E13, E14) so that they in sum issue the summed cathodic current to the tissue. More specifically, the downselector 108 is controlled to close the switching networks 140 at the anodic electrodes nodes 39 to connect them to a particular crossbar 117, and to close the switching networks 140 at the cathodic electrodes to connect them to another one of the crossbars 117. (The switching networks 140 are not shown in FIG. 12A for simplicity). In the example shown, S3ap, S4ap and Sap are asserted to connect the anodic electrode nodes at electrodes E3, E4, and E5 to crossbar ap; S13an and S14an are also asserted to connect the cathodic electrode nodes at electrodes E13 and E14 to crossbar an. (These anodic and cathodic electrode nodes could also be connected to any of the different crossbars bp, bn, cp, cn, etc.).

Note that when the downselector 108 is used to provide stimulation as in the bottom of FIG. 12A, the currents provided at the individual anodic and cathodic electrodes are not perfectly established at the prescribed currents. Instead, the currents provided at these electrodes will depend on the resistance of the patient's tissue. Nevertheless, the downselector 108 can be controlled to provide stimulation in manners that the stimulation circuitry 28 is not capable of, and so in conjunction with the stimulation circuitry 28 provides additional flexibility in providing stimulation.

When the downselector 108 is controlled during stimulation to assist in providing stimulation as just described, sensing is preferably disabled. This can occur by disabling the sense amps 110x during periods where stimulation is being provided (e.g., EN=‘0’, FIG. 6). The inputs to the sense amps can include optional switches which could be opened to decouple the inputs from the crossbars 117 to likewise disable sensing, as shown in dotted lines in FIG. 11. Additionally, the front-end of the sense amps 110a-d can include voltage protection circuitry to protect the sense amp inputs from high voltages that may appear at the electrode nodes (e.g., at the crossbars 117) during stimulation.

FIG. 12B shows a particular utility to use of the downselector 108 during stimulation. In this example, some of the electrodes on the lead (array 17) are directional, meaning that they span a particular number of degrees around the circumference of the lead. Electrodes E2-E4 for example each span roughly 120 degrees around the lead at the same longitudinal position along the axis of the lead; electrodes E5-E7 are similar, but are located at a different longitudinal position. Electrodes of this type are sometimes referred to as split-ring electrodes. Electrodes E1 and E8 by contrast are more conventional ring electrodes that span 360 degrees around the lead.

In the example of FIG. 12B, electrodes E2-E4 ideally each provide the same cathodic current (−2 mA, or −6 mA in total), with the case electrode Ec acting as a return and providing the sum of the anodic current (+6 mA). Providing stimulation in this manner might be particularly useful in an application in which the IPG 100 provides Deep Brain Stimulation (DBS) for example.

The top of FIG. 12B shows the traditional manner in which stimulation circuitry 28 would provide these currents to the tissue R without use of the downselector 108. The relevant PDACs or NDACs at the selected electrodes would be digitally programmed to produce the ideal amount of current (e.g., <In2>, <In3> and <In4> each provide −2 mA at electrodes E2, E3, and E4, while <IpC> provides +6 mA of current at electrode Ec). Because the downselector 108 is not active, all control signals to the various switching networks 140 are deasserted and all switching networks 140 are open.

The bottom of FIG. 12B shows an alterative method for approximately delivering this stimulation in a manner that involves control of the downselector 108. Here, one or more of the NDACs at the cathodic electrodes (e.g., NDAC2) is programmed to provide the desired summed cathodic current of −6 mA, and a single PDAC (PDACc) is programmed to provide the desired anodic current of +6 mA. The downselector 108 is then controlled to connect the cathodic electrodes nodes together so that they in sum issue the summed cathodic current to the tissue. More specifically, the downselector 108 is controlled to close the switching networks 140 at the cathodic electrodes nodes to connect them to a particular crossbar 117 to short these nodes together. In the example shown, S2ap, S3ap and S4ap are asserted to connect the electrode nodes at electrode E2, E3, and E4 to crossbar ap, although again another crossbar could be chosen. As explained earlier, the actual cathodic current provided at each of the cathodic electrodes (E2-E4) will depend on the resistance of the patient's tissue at each of these electrodes. Nevertheless, because the cathodic electrodes E2-E4 are similar, and located at the same longitudinal position with respect to the return electrode (Ec), it would be expected that the cathodic currents at each cathodic electrode would be roughly equal (i.e., −2 mA). As such, control of the downselector 108 in this fashion during stimulation allows directional electrodes at a common longitudinal position along the lead to be effectively driven as would a single ring electrode at the same longitudinal position.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A stimulator device, comprising:
a plurality of electrode nodes, wherein each of the electrode nodes is associated with a different electrode configured to contact a patient's tissue;
at least one sense amplifier comprising a first input and configured to sense a signal from the patient's tissue; and
a plurality of first switching networks each comprising
a first switch and a second switch connected in series between one of electrode nodes and the first input, and
a third switch connected between a reference voltage and a node between the first and second switches,
wherein each of the first switching networks is controllable by at least one first control signal.

2. The stimulator device of claim 1, wherein each at least one first control signal comprises a first signal and a second signal, wherein the second signal is a logical complement of the first signal.

3. The stimulator device of claim 2, wherein the first signal controls the first and second switches, and wherein the second signal controls the third switch.

4. The stimulator device of claim 2, wherein the first switch comprises a first transfer gate, and wherein the second switch comprises a second transfer gate.

5. The stimulator device of claim 4, wherein the first and second transfers gates each comprise a N-channel and P-channel transistor connected in parallel.

6. The stimulator device of claim 5, wherein the first signal controls the N-channel transistors in the first and second transfer gates, and wherein the second signal controls the P-channel transistors in the first and second transfer gates.

7. The stimulator device of claim 6, wherein the second signal also controls and the third switch.

8. The stimulator device of claim 1, wherein the reference voltage comprises ground.

9. The stimulator device of claim 1, wherein each first switching network is configured so that the first and second switches are closed at the same time that the third switch is opened, and the first and second switches are opened at the same time that the third switch is closed.

10. The stimulator device of claim 1, wherein the at least one first control signals are issued by control circuitry.

11. The stimulator device of claim 10, wherein the control circuitry is powered by a first power supply voltage.

12. The stimulator device of claim 11, wherein the at least one first control signals are established in a second domain comprising a second power supply voltage higher than the first power supply voltage.

13. The stimulator device of claim 12, further comprising level translator circuitry to produce the at least one first control signals from control signals established in a first domain comprising the first power supply voltage.

14. The stimulator device of claim 12, further comprising a boost circuit configured to generate a boosted power supply voltage higher than the first power supply voltage.

15. The stimulator device of claim 14, wherein the second power supply voltage comprises the boosted power supply voltage.

16. The stimulator device of claim 12, further comprising stimulation circuitry configured to issue stimulation to the plurality of electrode nodes, wherein the stimulation circuitry is powered by a compliance power supply voltage.

17. The stimulator device of claim 16, further comprising a selector circuit configured to establish the second power supply voltage at a higher of the boosted and compliance power supply voltages.

18. The stimulator device of claim 1, wherein the at least one sense amplifier comprises a second input, and further comprising
a plurality of second switching networks each comprising
a first switch and a second switch connected in series between the second input and either (i) one of the electrode nodes, or (ii) a DC voltage, and
a third switch connected between a reference voltage and a node between the first and second switches,
wherein each of the second switching networks is controllable by at least one second control signal.

19. The stimulator device of claim 18, further comprising control circuitry configured to issue the at least one first control signal and the at least one second control signal.

20. The stimulator device of claim 19, wherein the control circuitry is configured to issue the at least one first control signal and the at least one second control signal to couple different of the electrode nodes to the first and second inputs, or to couple one of the electrode nodes to the first input and the DC voltage to the second input.
